(19) United States
(12) Patent Application Publication
(10) Pub. No.: US 2014/0161852 A1
(43) Pub. Date: Jun. 12, 2014

(54) NANOPARTICLE DENTAL COMPOSITION AND METHOD OF MAKING

(71) Applicants: Yangfang Ren, Rochester, NY (US); Qin Amy Wang, Rochester, NY (US); Hans Malmstrom, Rochester, NY (US)

(72) Inventors: Yangfang Ren, Rochester, NY (US); Qin Amy Wang, Rochester, NY (US); Hans Malmstrom, Rochester, NY (US)

(73) Assignee: UNIVERSITY OF ROCHESTER, Rochester, NY (US)

(21) Appl. No.: 14/113,132

(22) PCT Filed: Apr. 20, 2012
(86) PCT No.: PCT/US12/34439

§ 371 Date: Feb. 19, 2014

Related U.S. Application Data
(60) Provisional application No. 61/477,261, filed on Apr. 20, 2011.

(51) Int. Cl. A61K 8/24; A61Q 11/00; A61K 8/21

(52) U.S. Cl. CPC A61K 8/24; A61K 8/21; A61Q 11/00
USPC 424/401; 424/52; 423/301; 423/490; 428/402

(57) ABSTRACT

Disclosed are compositions and methods for reducing dentin hypersensitivity. Also disclosed are compositions containing calcium fluoride nanoparticles.

### BACKGROUND

1. Field of the Invention

The present disclosure is directed to compositions suitable for use in dental applications, and specifically to compositions suitable for use in the treatment of sensitive teeth, together with methods for preparing the same.

2. Technical Background

Dentine hypersensitivity is a condition that affects a significant portion of the population. Those afflicted with dentine hypersensitivity can experience irritation and/or pain upon oral exposure to, for example, hot or cold substances. Dentine hypersensitivity can occur when nerves inside the dentin of the teeth are exposed to changing environmental conditions.

Dentin is a material disposed between the pulp and enamel layers of the crown as well as between the cementum and pulp of the root of a tooth. Dentin contains a large number of microscopic tubular structures, called dentinal tubules, which are typically about 0.5 to 2 micrometers in diameter and extend radially through the dentin, from the pulp to the enamel. Contained within the dentinal tubules is a plasma-like fluid. Changes in the pressure or flow of this fluid can occur from environmental stimuli, such as heat, cold, exposure to certain substances, or mechanical pressure (e.g., from brushing), and can generate pain responses from nerve endings at the pulp end of the tubules.

In healthy teeth, the outermost end of the dentin tubules are typically covered by a layer of cementum or enamel, and are thus not exposed directly to the oral cavity. Erosion of enamel or cementum can expose the tubules directly to, for example, hot or cold substances in the mouth, increasing the chance of pain or sensitivity.

Various methods have been explored to reduce tooth sensitivity with limited success. Potassium compounds, such as potassium nitrate, have been used to reduce the excitability of nerves in teeth. Blocking agents, such as hectorite and montmorillonite clays have also been used to block or plug the tubules. Water-soluble or water-swellable polymers and polyelectrolytes have also been used to physically block tubules and prevent movement of the fluid therein. To date, none of these methods have been able to provide a long-lasting and effective reduction in tooth sensitivity. Thus, a need exists for treatment compositions and methods that can provide a long-term reduction in tooth sensitivity. These needs and other needs are at least partially satisfied by the present invention.

### SUMMARY

In accordance with the purpose(s) of the invention, as embodied and broadly described herein, the disclosure, in one aspect, relates to compositions suitable for use in dental applications, and specifically to compositions suitable for use in the treatment of sensitive teeth, together with methods for preparing the same.

In one aspect, the present disclosure provides a composition comprising calcium fluoride nanoparticles.

In another aspect, the present disclosure provides a composition comprising calcium fluoride phosphate nanoparticles.

In another aspect, the present disclosure provides a nanocomposite comprising calcium fluoride nanoparticles and at least one of dicalcium phosphate nanoparticles, Hydroxyapatite nanoparticles, or a combination thereof.

In another aspect, the present disclosure provides a dentrifice comprising calcium, phosphate, and fluoride ions.

In yet another aspect, the present disclosure provides a dentrifice capable of occluding at least a portion of exposed dentin tubules when topically applied to a tooth surface.

In another aspect, the present disclosure provides a method for reducing dentin hypersensitivity, the method comprising contacting a composition comprising calcium fluoride nanoparticles with a tooth surface.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 illustrates field emission scanning electron micrographs (FESEM), transmission electron micrographs (TEM), and size distribution data for calcium fluoride nanoparticles produced from various reactions, all in accordance with various aspects of the present disclosure as described below:
20 mM CaCl₂/40 mM NH₄F: (A1) FESEM image, (A2) TEM image and (B) size distribution;
150 mM CaCl₂/300 mM NH₄F: (C1) FESEM image, (C2) TEM image, and (D) size distribution;
0.5 M CaCl₂/1.0 M NH₄F: (E) FESEM image and (F) size distribution;
1.0 M CaCl₂/2.0 M NH₄F: (G) FESEM image, (H1-2) size distribution;
0.5 M CaCl₂/1.0 M NaF: (I) FESEM image, (J) size distribution; and
(K) energy dispersive X-ray spectroscopy (EDS), and (L) X-ray diffraction analysis (XRD).

FIG. 2 illustrates characterization data for Hydroxyapatite nanoparticles (nanoHA), prepared in accordance with various aspects of the present disclosure: (A) FESEM image of agglomerated rod-like and flake structures, (B1, B2) size distribution of the agglomerated particles, (C) comparison of XRD patterns for inventive nanoHA and commercially available nanoHA, and (D) EDS pattern of inventive nanoHA.

FIG. 3 illustrates electron micrographs of: (A) pretreated dentin tubules, (B1) completely and (B2) partially covered dentin disk surface from a single treatment, in accordance with various aspects of the present disclosure; and (C1) completely and (C2) partially covered dentin disk surface. FIG. 3 (D1) illustrates a completely covered and (D2) partially covered dentin disk surface from a single treatment with commercially available PERMASEAL®, a methacrylate based resin. FIG. 3(E) illustrates completely occluded and (F) partially occluded dentin tubules at high magnification, and (G) elemental analysis by EDS.

FIG. 4 illustrates electron micrographs of a dentin disk after a single treatment with a calcium fluoride nanoparticles gel: (A) pretreated dentin tubules, (B1, B2) after a single treatment, (C1, C2) after a single treatment and immersion in human saliva for 24 hours, (D1, D2) after a single treatment with 100 minutes in Coca-Cola®, and high magnification micrographs of (E) completely and (F) partially occluded dentin tubules.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates.

As used in the specification and the appended claims, the singular forms “a,” “an” and “the” include plural referents unless the context clearly dictates otherwise. Thus, for example, reference to “a calcium compound” includes mixtures of two or more such calcium compounds.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or can not occur, and that the description includes instances where said event or circumstance occurs and instances where it does not.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As briefly described above, the present invention relates to compositions and methods that can be useful, for example, in reducing tooth sensitivity. In one aspect, the invention comprises calcium fluoride nanoparticles. In another aspect, the invention comprises a composite of calcium fluoride nanoparticles and at least one other nanomaterial. In yet another aspect, the invention comprises methods for preparing such compositions comprising calcium fluoride nanoparticles. In still other aspects, the invention comprises treatment compositions, such as, for example, suspensions and gels comprising calcium fluoride nanoparticles. In other aspects, the invention comprises methods for utilizing the inventive compositions, such as, for example, methods for treating a subject so as to reduce tooth sensitivity.

In one aspect, the invention comprises calcium fluoride nanoparticles. In another aspect, the invention comprises calcium fluoride nanoparticles and at least one of dicalcium phosphate nanoparticles, hydroxyapatite nanoparticles, or a combination thereof. In another aspect, the invention comprises calcium fluoride phosphate nanoparticles.

### Synthesis of Calcium Fluoride Nanoparticles

In one aspect, calcium fluoride nanoparticles (nanoCaF₂) can be prepared by contacting a calcium containing compound, such as, for example, calcium chloride, and a fluorine containing compound, such as, for example, ammonium fluoride or sodium fluoride. In one aspect, each of the calcium containing compound and the fluorine containing compound are contacted in the form of a dilute solution. In another aspect, at least one of the calcium containing compound or the fluorine-containing compound is contacted in the form of a dilute aqueous solution. In another aspect, both of the calcium containing compound and the fluorine containing compound are contacted in the form of a dilute aqueous solution. In yet another aspect, non-aqueous or mixed solvent systems can be utilized for either or both of the calcium containing compound and the fluorine containing compound, provided that the reactants can mix so as to form a desired calcium fluoride nanoparticle. Calcium containing compounds and fluorine containing compounds, such as those recited herein, are commercially available. One of skill in the art could, in possession of this disclosure, readily select appropriate calcium containing compound and/or fluorine containing compound for use in preparing the inventive calcium fluoride nanoparticles.

In another aspect, the calcium containing compound can comprise any calcium containing compound capable of provide a calcium ion. In a specific aspect, the calcium containing compound comprises calcium chloride. In other aspects, the calcium containing compound can comprise a mixture of two or more calcium containing compounds. In various aspects, a calcium ion in the one or more calcium containing compounds can exhibit the same or varying oxidation states, and the calcium containing compounds can exhibit varying purity levels.

Similarly, the fluorine containing compound can comprise any suitable compound capable of provide a fluoride ion. In one aspect, the fluorine containing compound comprises ammonium fluoride. In another aspect, the fluorine containing compound comprises sodium fluoride. In yet another aspect, the fluorine containing compound can comprise a mixture of two or more fluorine containing compounds.

In one aspect, the calcium containing compound and the fluorine-containing compound are contacted with continuous or substantially continuous mixing. In various aspects, such mixing can occur via stirring, agitation, shaking, or other methods that one of skill in the art may deem appropriate. In a specific aspect, the calcium containing compound and the fluorine containing compound are mixed under constant stirring conditions. In one aspect, the particular speed and/or degree of mixing can vary.

In one aspect, the concentration of the calcium containing compound and/or the fluorine containing compound can be any concentration suitable for producing nano-sized calcium fluoride particles. In various aspects, the concentration of a calcium containing compound can be from about 1 mM to about 4 M, for example, about 1 mM, 2 mM, 3 mM, 4 mM, 5 mM, 8 mM, 10 mM, 15 mM, 20 mM, 25 mM, 50 mM, 75 mM, 100 mM, 150 mM, 200 mM, 250 mM, 0.5 M, 0.75 M, 1 M, 1.5 M, 2 M, 2.5 M, 3 M, 3.5 M, or 4 M; from about 1 mM to about 1M, for example, about 1 mM, 2 mM, 3 mM, 4 mM, 5 mM, 8 mM, 10 mM, 15 mM, 20 mM, 25 mM, 50 mM, 75 mM, 100 mM, 150 mM, 200 mM, 250 mM, 0.5 M, 0.75 M, or 1 M; from about 1 mM to about 0.5 M, for example, about 1 mM, 2 mM, 3 mM, 4 mM, 5 mM, 8 mM, 10 mM, 15 mM, 20 mM, 25 mM, 50 mM, 75 mM, 100 mM, 150 mM, 200 mM, 250 mM, or 0.5 M; or from about 1 mM to about 250 mM, for example, about 1 mM, 2 mM, 3 mM, 4 mM, 5 mM, 8 mM, 10 mM, 15 mM, 20 mM, 25 mM, 50 mM, 75 mM, 100 mM, 150 mM, or 200 mM. In a preferred aspect, the calcium containing compound is provided and contacted in the form of an aqueous solution having a concentration of from about 25 mM to about 0.5 M.

Similarly, the concentration of a fluorine containing compound can, in various aspects, be from about 1 mM to about 4 M, for example, about 1 mM, 2 mM, 3 mM, 4 mM, 5 mM, 8 mM, 10 mM, 15 mM, 20 mM, 25 mM, 50 mM, 75 mM, 100 mM, 150 mM, 200 mM, 250 mM, 0.5 M, 0.75 M, 1 M, 1.5 M, 2 M, 2.5 M, 3 M, 3.5 M, or 4 M; from about 1 mM to about 1M, for example, about 1 mM, 2 mM, 3 mM, 4 mM, 5 mM, 8 mM, 10 mM, 15 mM, 20 mM, 25 mM, 50 mM, 75 mM, 100 mM, 150 mM, 200 mM, 250 mM, 0.5 M, 0.75 M, or 1 M; from about 1 mM to about 0.5 M, for example, about 1 mM, 2 mM, 3 mM, 4 mM, 5 mM, 8 mM, 10 mM, 15 mM, 20 mM, 25 mM, 50 mM, 75 mM, 100 mM, 150 mM, 200 mM, 250 mM, or 0.5 M; or from about 1 mM to about 250 mM, for example, about 1 mM, 2 mM, 3 mM, 4 mM, 5 mM, 8 mM, 10 mM, 15 mM, 20 mM, 25 mM, 50 mM, 75 mM, 100 mM, 150 mM, or 200 mM. In a preferred aspect, the fluorine containing compound is provided and contacted in the form of an aqueous solution having a concentration of from about 25 mM to about 0.5 M. In still other aspects, the calcium containing compound, the fluorine containing compound, or both, can be provided or contacted in a concentration less than or greater than any values specifically recited herein, and the present invention is not intended to be limited to any particular concentration value or range.

In one aspect, the calcium containing compound and the fluorine containing compound can be contacted such that the molar ratio of calcium to fluoride is from about 0.3 to about 0.7, for example, about 0.3, 0.4, 0.5, 0.6, or 0.7. In a specific aspect, the molar ratio of calcium to fluoride is about 0.5, such that about 0.5 moles of calcium containing compound is contacted with about 1 mole of a fluorine-containing compound. In another aspect, the fluoride containing compound, such as, for example, sodium fluoride, ammonium fluoride, or a combination thereof, can be added to the calcium containing compound in a dropwise manner over a period of time.

In various aspects, the yield of calcium fluoride nanoparticles can be at least about 30%, at least about 35%, at least about 40%, or greater. In one aspect, the yield of calcium fluoride nanoparticles can be from about 32.9% to about 38.5%, for example, about 33, 34, 35, 36, 37, or 38%.

In one aspect, the resulting nanoparticles can be recovered by, for example, centrifuging. After recovery, the nanoparticles can be purified by washing at least once in distilled and/or deionized water. In another aspect, the recovered nanoparticles can be washed multiple times in distilled and/or deionized water. For storage, the purified nanoparticles can optionally be frozen and lyophilized for later use.

In one aspect, the calcium fluoride nanoparticles can have an average particle size of from about 10 nm to about 150 nm, for example, about 10, 15, 20, 25, 30, 35, 40, 45, 50, 55, 60, 65, 70, 75, 80, 85, 90, 100, 110, 120, 130, 140, or 150 nm. In another aspect, at least a portion of the calcium fluoride nanoparticles have an average particle size of less than about 60 nm. In yet another aspect, at least a portion of the calcium fluoride nanoparticles have an average particle size of from about 25 nm to about 60 nm. It should be noted that particle size is a distributional property and that at least a portion of any particular sample of calcium fluoride nanoparticles can be larger than or smaller than the average particle size. In another aspect, at least a portion of the calcium fluoride nanoparticles can have a size less than 10 nm or greater than 150 nm, and the present invention is not intended to be limited to any particular particle size and/or distribution. While not wishing to be bound by theory, particle size generally increases with increasing concentration of the calcium containing compound and/or fluorine containing compound.

The morphology of the produced calcium fluoride nanoparticles can vary, for example, from spherical to oblong. In one aspect, spherical or substantially spherical nanoparticles are typically produced at lower reactant concentrations, whereas oblong or football-like nanoparticles are can be produced at higher reactant concentrations.

In another aspect, the calcium fluoride nanoparticles can form agglomerates, ranging in size from about 50 nm to about 400 nm, for example, about 50, 75, 100, 125, 150, 175, 200, 225, 250, 275, 300, 325, 350, 375, or 400 nm. In other aspects, agglomerates, if present, can be smaller or larger than the values recited herein, and the present disclosure is not intended to be limited to any particular agglomerate size.

In one aspect, calcium fluoride nanoparticles produced by the various methods of the present invention are pure or substantially pure calcium fluoride. In another aspect, the purity of any produced calcium fluoride nanoparticles can vary, based on, for example, the purity of the reactants.

In a specific aspect, a dilute ammonium fluoride solution is slowly added (e.g., dropwise) to a dilute calcium chloride solution under constant stirring. In another specific aspect, a dilute sodium fluoride solution is slowly added to a dilute calcium chloride solution under constant stirring.

### Synthesis of Anhydrous Dicalcium Phosphate Nanoparticles

The dicalcium phosphate nanoparticles of the present invention can, in one aspect, be prepared by contacting a quantity of dicalcium phosphate, such as, for example, in aqueous solution form, with an acid, such as for example, acetic acid. In one aspect, the dicalcium phosphate is anhydrous. In another aspect, the acid comprises one or more organic acids. In a specific aspect, the acid comprises acetic acid. In another aspect, other organic acids or mixtures of acids can be used and the present disclosure is not intended to be limited to any particular acid. In another aspect, the acetic acid and/or dicalcium phosphate solution are stirred during contacting. In one aspect, the amount of anhydrous dicalcium phosphate contacted is an amount sufficient to produce a solution having concentration of from about 0.5 M to about 1.5 M. In another aspect, the amount of dicalcium phosphate can vary, and the present invention is not intended to be limited to any particular concentration. In one aspect, the acetic acid can have a concentration of from about 0.5 to about 2.5 M. In a specific aspect, the acetic acid is about 1.6 M. In other aspects, the acetic acid can have a concentration lower than or greater than any value specifically recited herein, and the present invention is not intended to be limited to any particular concentration.

After contacting the dicalcium phosphate with the acetic acid, the solution can optionally be continuously stirred for a period of time, for example, up to about 5 days or more. In another aspect, the resulting solution can then optionally be sonicated to disperse the particles in the solution. In one aspect, the resulting solution is sonicated for about 10 minutes. In one aspect, the pH of the resulting solution is maintained at a pH of from about 3.6 to about 3.9 after contacting. In another aspect, the pH of the resulting solution, after contacting, can be adjusted so as to remain from about 3.6 to about 3.9.

The dicalcium phosphate particles can be purified by washing one or multiple times with distilled and/or deionized water. Once purified, the dicalcium phosphate particles can be frozen and/or lyophilized for storage or future use.

### Synthesis of Hydroxyapatite Nanoparticles

In one aspect, Hydroxyapatite nanoparticles can be prepared by contacting a CaCl₂ solution, such as, for example, about 44.3 mM CaCl₂, with a H₃PO₄ solution, for example, about 26.6 mM H₃PO₄. In another aspect, a 443 mM CaCl₂ solution can be contacted with a 266 mM H₃PO₄ solution. In yet another aspect, a 1 M CaCl₂ solution can be contacted with a 0.6 M H₃PO₄ solution. In yet another aspect, a 1 M Ca(OH)₂ solution can be contacted with a 0.6 M H₃PO₄ solution. In still another aspect, a 1 M CaCl₂ solution can be contacted with a 0.6 M KH₂PO₄ solution. In still another aspect, a 1 M CaCl₂ solution can be contacted with a 0.6 M Na₂HPO₄ solution. In another aspect, the molar ratio of calcium (Ca) to phosphate (PO₄) is about 1.67. In still other aspects, the pH can be maintained at between about 9 and about 12 during the course of the reaction. In another aspect, nanoHA particles can be prepared by precipitation using, for example, continuous stirring methods for about 7 days, and/or precipitation under continuous stirring methods for about 3 days followed by ultrasonic dispersion. In one aspect, a combination of methods can be used for preparing nanoHA particles. In still another aspect, after synthesis, all or a portion of the nanoHA particles can be purified and/or lyophilized.

In one aspect, the yield of Hydroxyapatite nanoparticles (nanoHA) from the reaction of calcium hydroxide and phosphoric acid can be at least about 50%. In a specific aspect, the yield can be about 62%. In one aspect, at least a portion of the produced nanoHA particles had a rod-like morphology. In another aspect, at least a portion of the produced nanoHA particles had a flake-like morphology.

In one aspect, the size of rod-like nanoHA particles can be from about 100 nm to about 160 nm in length, and from about 10 nm to about 30 nm in width. In another aspect, the size of flake-like nanoHA particles can be from about 200 nm to about 400 nm. In another aspect, at least a portion of the nanoHA particles can be agglomerated, having an agglomerate size of from about 90 nm to about 900 nm in length, and from about 40 nm to about 300 nm in width.

In one aspect, at least a portion of the nanoHA particles can comprise hydroxylfluoroxyapatite and/or calcium hydrogen phosphate hydroxide. In one aspect, the preparation of nanohydroxyapatite particles does not comprise a fluorine containing compound.

### Synthesis of Calcium Fluoride Phosphate (Fluoroapatite) Nanoparticles

In one aspect, calcium fluoride phosphate particles can be prepared by contacting a calcium containing compound, a fluorine containing compound, and a phosphate containing compound. In various aspects, the calcium containing compound and/or the fluorine containing compound can be the same or substantially the same as those described herein with respect to the synthesis of calcium fluoride nanoparticles. In various specific aspects, the calcium containing compound can comprise calcium hydroxide, calcium chloride, or a combination thereof. In other specific aspects, the fluorine containing compound can comprise ammonium fluoride, sodium fluoride, or a combination thereof.

The phosphate containing compound can comprise any compound capable of providing phosphate and that can result in the production of a calcium fluoride phosphate nanoparticle, after contacting with the calcium containing compound, the fluorine containing compound, and any other optional reactants. In one aspect, the phosphate containing compound comprises phosphoric acid. In one aspect, the molar ratio of calcium to phosphorus (i.e., in reactants) is about 1.0 to 2.0, preferably about 1.67. In another aspect, the molar ratio of calcium to fluorine (i.e., in reactants) is from about 0.25 to about 1.5, preferably about 0.5 to 1.0.

In one aspect, a quantity of dilute phosphoric acid (e.g., about 0.6 M) is added slowly, for example, dropwise, to an aqueous solution of calcium hydroxide while stirring. Simultaneous to and/or subsequent to the phosphoric acid addition, a quantity of sodium fluoride (e.g., about 1 M) can be added to the calcium hydroxide solution. In one aspect, the reaction can be allowed to continue for a period of time, for example, about 7 days, while maintaining the pH between about 9 and about 12, for example, about 12.0.

In another aspect, a quantity of dilute phosphoric acid (e.g., about 0.6 M) is added slowly, for example, dropwise, to an aqueous solution of calcium chloride while stirring. Simultaneous to and/or subsequent to the phosphoric acid addition, a quantity of ammonium fluoride (e.g., about 2 M) is added to the calcium chloride solution, and a quantity of sodium hydroxide (e.g., 10 N) added to adjust the pH to about 12.0. In one aspect, the reaction can be allowed to continue for a period of time, for example, about 7 days, while maintaining the pH at about 12.0.

After contacting for a period of time, the calcium fluoride phosphate nanoparticles can optionally be purified by washing one or multiple times with distilled and/or deionized water. The purified calcium fluoride phosphate nanoparticles can also optionally be frozen and lyophilized for storage or later use.

### Preparation of Composite

In one aspect, calcium fluoride nanoparticles or fluoroapatite nanoparticles, as described herein, can be utilized alone. In another aspect, calcium fluoride nanoparticles can be utilized together with at least one of dicalcium phosphate nanoparticles, hydroxyapatite nanoparticles, or a combination thereof.

In one aspect, a composite mixture (nanoCaF₂/nanoDCPA) can be prepared by mixing calcium fluoride nanoparticles and dicalcium phosphate nanoparticles. In another aspect, a composite mixture (nanoCaF₂/nanoHA) can be prepared by mixing calcium fluoride nanoparticles and Hydroxyapatite nanoparticles. In various aspects, a composite mixture of nanoCaF₂/nanoDCPA can have a ratio of about 1:1, about 2:1, or about 3:2. In other aspects, the ratio of nanoCaF₂:nanoDCPA can vary, and the present invention is not intended to be limited to any particular ratio.

### Applications

In one aspect, any of the nanoparticle compounds or composites described herein can be utilized alone or in combination with other materials. For example, the calcium fluoride nanoparticles or a composite containing calcium fluoride nanoparticles can be utilized with processing aids, extenders, and/or other dental compatible materials. In one aspect, the inventive materials can comprise a portion of a dentifrice, such as a liquid, powder, or gel. In one aspect, the inventive materials can be utilized as a component in toothpaste or other oral care composition. In another aspect, the inventive materials can comprise a suspension, paste and/or gel. In other aspects, the inventive materials can comprise or be a part of a bonding agent, a sealing agent, or other carrier. In various aspects, compositions comprising the inventive materials can also comprise materials such as sealers, varnishes, luting agents, desensitizing agents, adhesive polymers, and/or carriers. Thus, in one aspect, the disclosure provides a dentifrice comprising any one or more of the nanoparticles and/or composites of nanoparticles described herein.

In one aspect, the inventive materials can be utilized as a portion of a home dental care kit, wherein a subject can apply, for example, topically, the inventive materials to teeth so as to reduce sensitivity. In such an aspect, the inventive material can be a portion of toothpaste that can be brushed onto teeth. When the teeth are brushed, the inventive nanoparticles can infiltrate exposed dentin tubules. In another aspect, the inventive materials can be utilized as a portion of a treatment to be applied by, for example, a dental care professional.

### Treatment and Reduction in Sensitivity

In one aspect, application of the inventive materials, for example, topically, to all or a portion of a subject's teeth can reduce sensitivity to environmental stimuli as described herein. In another aspect, application of the inventive materials can at least partially occlude exposed dentin tubules, thereby reducing sensitivity to environmental stimuli. In such an aspect, the deposited nanoparticles can narrow or block exposed dentin tubules. In another aspect, application of the inventive materials can at least partially remineralize teeth by providing elemental components necessary for such remineralization. Such remineralization can, in various aspects, increase resistance to erosion by, for example, acidic foods and beverages.

In yet another aspect, and in contrast to conventional sealants that merely coat the exterior of a tooth, the nanoparticles of the present invention can provide better infiltration into dentin tubules than other approaches and materials. In still another aspect, the nanoparticles of the present invention can provide better adhesion to dentin and enamel surfaces than other materials. Use of the inventive materials on teeth having exposed dentin tubules can, in various aspects, provide permanent occlusion of dentin tubules, together with remineralization of tooth enamel. In another aspect, the benefit achieved by use of the inventive materials can provide extended reduction in hypersensitivity as compared to other commercially available treatments and approaches.

In various aspects, the preparation methods of the present invention are easily scalable without requiring expensive equipment or control systems. As such, high production rates can be achieved at reasonable costs.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Synthesis of Calcium Fluoride Nanoparticles—Method A

In a first example, nanoparticles of calcium fluoride were synthesized by precipitation using a continuous stirring method. The molar ratio of calcium to fluoride was 0.5. Initially, 200 ml of 1.0 M ammonium fluoride (NH₄F) was added to 200 ml of 0.5 M calcium chloride (CaCl₂) under continuously magnetic stirring for 24 hours at room temperature, according to the reaction scheme below.

CaCl₂+2NH₄F=CaF₂+2NH₄Cl (1)

The resulting nanoparticles were purified by washing with deionized water after centrifuging at 13,000 rpm for 10 minutes. A portion of the purified nanoparticles was then dispersed in 5-10 ml of deionized water, frozen at −80° C. overnight, and lyophilized for 24-48 hours.

2. Synthesis of Calcium Fluoride Nanoparticles—Method B

In a second example, nanoparticles of calcium fluoride were synthesized by precipitation using a continuous stirring method. The molar ratio of calcium to fluoride was 0.5. Initially, 200 ml of 1.0 M sodium fluoride was added to 200 ml of 0.5 M calcium chloride (CaCl₂) under continuously magnetic stirring for 24 hours at room temperature, according to the reaction scheme below.

CaCl₂+2NaF=CaF₂+NaCl (2)

The resulting nanoparticles were purified by washing with deionized water after centrifuging at 13,000 rpm for 10 minutes. A portion of the purified nanoparticles was then dispersed in 5-10 ml of deionized water, frozen at −80° C. overnight, and lyophilized for 24-48 hours.

3. Characterization of Calcium Fluoride Nanoparticles

Characterization results from the calcium fluoride nanoparticles prepared in Examples 1 and 2, above, are detailed in Table 1. The morphology and homogeneity of the resulting nanoparticles were determined by field emission scanning electron microscopy (FESEM) and transmission electron microscopy (TEM). Particle sizes were calculated using ImageJ software and are represented as a mean value±a standard deviation (nm). Elemental analysis and phased determination were determined using energy dispersive X-ray spectroscopy (EDS or EDX) and X-ray diffraction spectroscopy (XRD), respectively.

The yield rate of nanoparticles from reaction scheme (1) was from 32.9% to 38.5%. FESEM images demonstrated that the morphology of calcium fluoride nanoparticles changed from spherical (see FIG. 1A1, 1C1, 1E) to football-like (see FIG. 1G) when the concentrations of CaCl₂ and NH₄Cl were increased.

The CaF₂ nanoparticles were agglomerated to form nanoclusters ranging from about 68 nm to about 312 nm. TEM images at high magnification confirmed this finding (see FIG. 1A2 and 1C2). The average size of calcium fluoride nanoparticles also increased from 37±8 nm to 95±22 nm (L)/59±14 nm (W) when the reactant concentrations increased.

In reaction scheme (2), the morphology of resulting nanoparticles was spherical (see FIG. 1I) and the average size was about 28±9 nm with a range of 14-50 nm. The nanoparticles were also agglomerated similar to those from reaction scheme (1). The size distributions illustrate that CaF₂ nanoparticles from both reaction schemes were homogeneous. Similarly, EDS and XRD analysis confirmed that the particles were comprised of pure CaF₂.

TABLE 1
(Comment: I modified the space of this table)

| | Mean | Range | | XRD |
|---|---|---|---|---|
| Reaction | (nm) | (nm) | Morphology | Phase |
| 20 mM CaCl₂/ | 37 ± 8 | 18-66 | Spherical | CaF₂ |
| 40 mM NH₄F | | | | |
| 150 mM CaCl₂/ | 46 ± 19 | 19-98 | Spherical | CaF₂ |
| 300 mM NH₄F | | | | |
| 0.5M CaCl₂/ | 62 ± 18 | 34-128 | Spherical, | CaF₂ |
| 1.0M NH₄F | | | Football-like | |
| 1.0M CaCl₂/ | W: 59 ± 14 | 27-97 | Football-like | CaF₂ |
| 2.0M NH₄F | L: 95 ± 22 | 52-166 | | |
| 0.5M CaCl₂/ | 28 ± 9 | 14-50 | Spherical | CaF₂ |
| 1.0M NH₄F | | | | |

4. Preparation of Anhydrous Dicalcium Phosphate Nanoparticles

In a fourth example, anhydrous dicalcium phosphate nanoparticles were prepared by adding 21.77 g of anhydrous dicalcium phosphate to 200 ml of 1.6 M acetic acid under constant stirring at room temperature for five days, followed by sonication for 10 minutes. The pH was then adjusted to about 3.6 to 3.9 using HCl and/or NaOH as needed. The resulting nanoparticles were then purified by washing 3 times with deionized water, and then frozen and lyophilized as described in Example 1.

5. Synthesis of Hydroxyapatite Nanoparticles

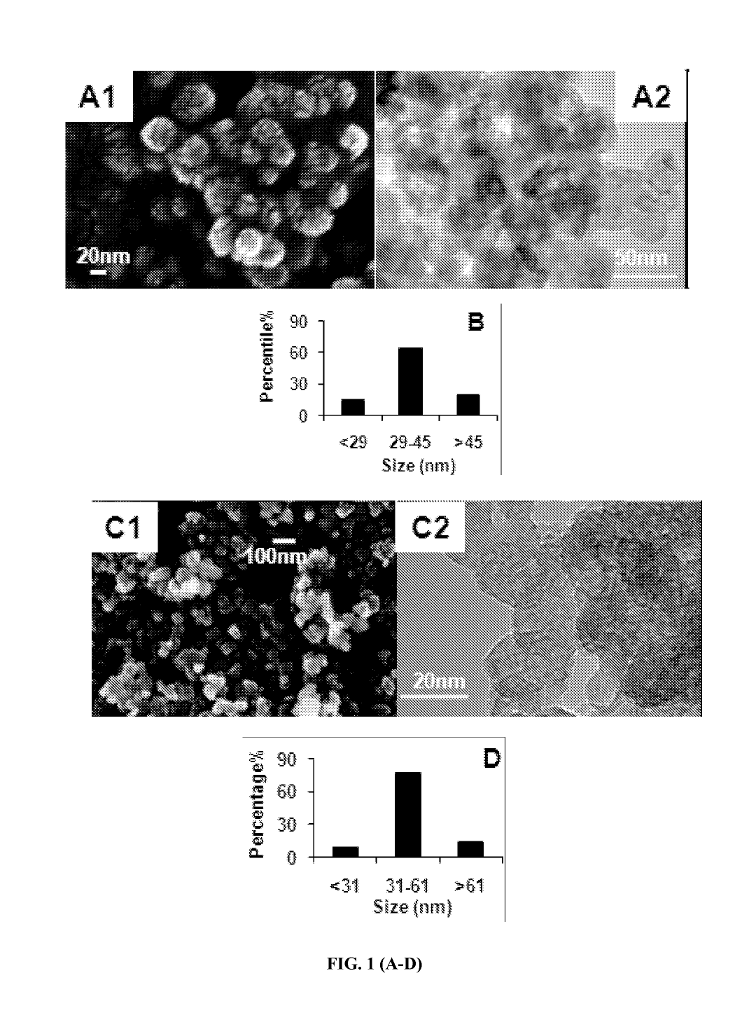

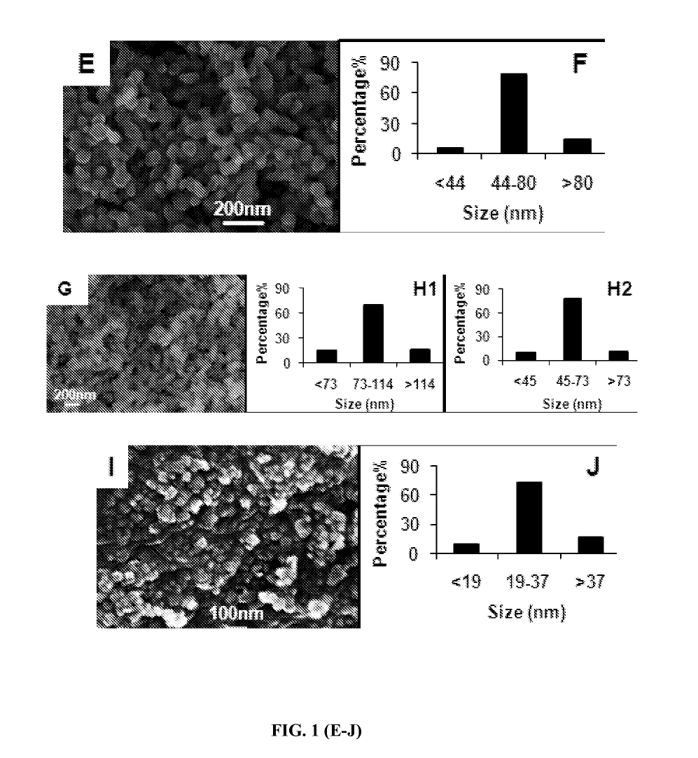

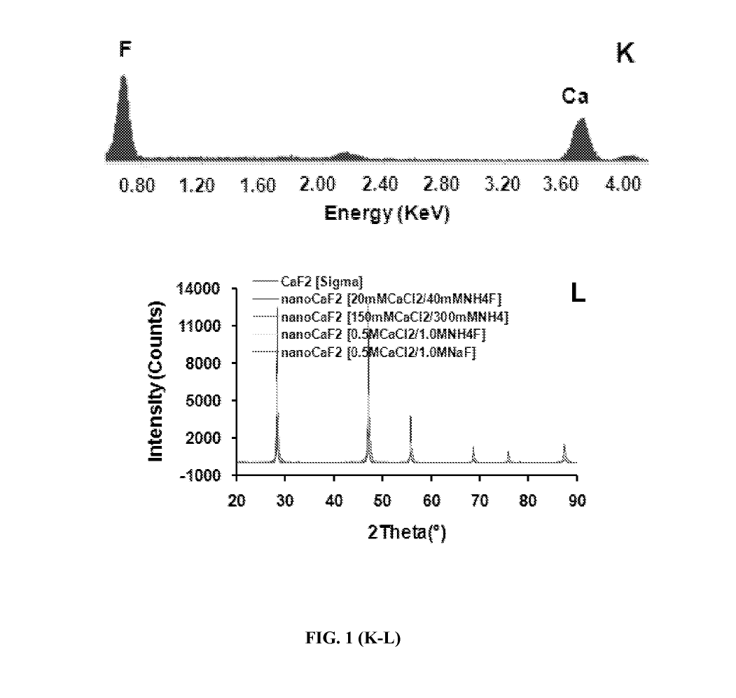

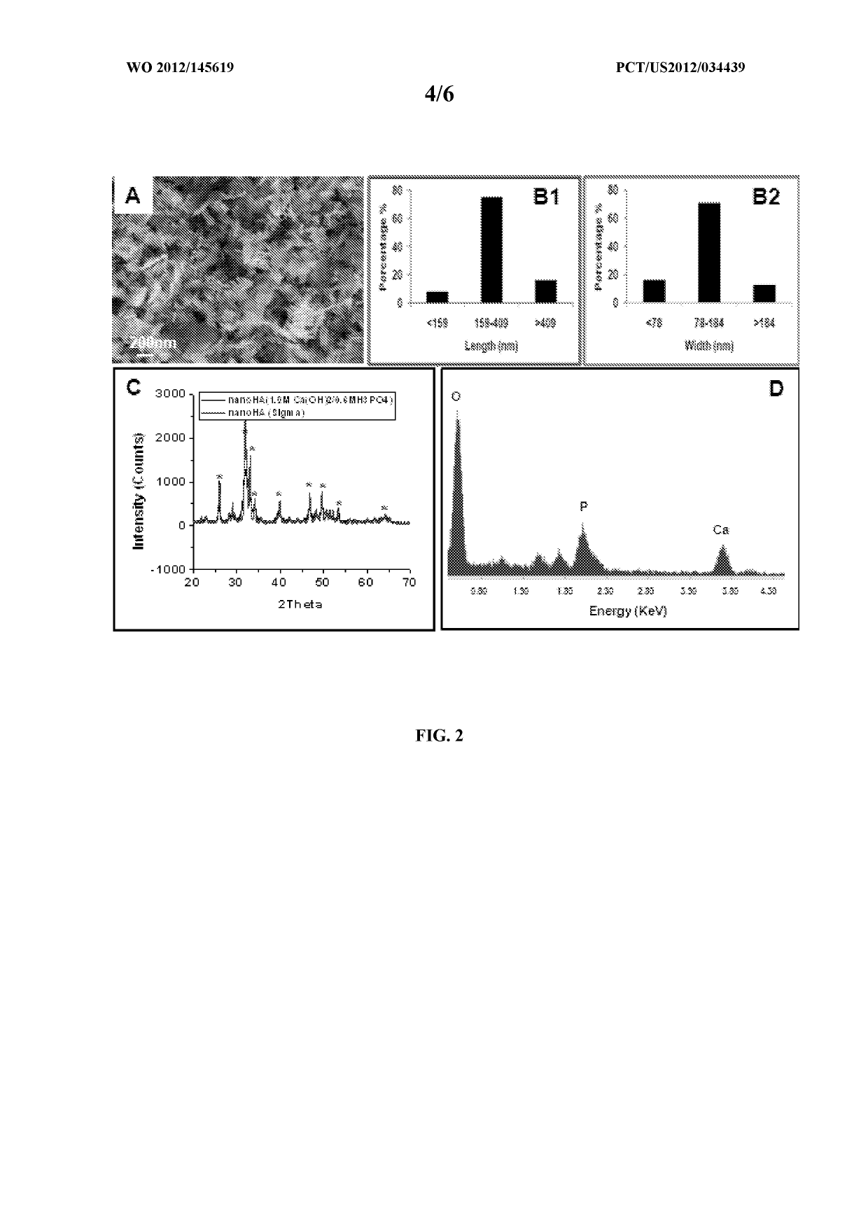

In a fifth example, Hydroxyapatite nanoparticles (nanoHA) were synthesized by a double step stirring method from the reaction of Ca(OH)₂ and H₃PO₄. The yield rate from the reaction was 61.9%. Analysis by FESEM of the resulting nanoHA particles indicated rod-like particles (see FIG. 2A) with an average size of 128±28 nm in length and 18±7 nm in width (see FIG. 2B1, 2B2). Some of the nanoHA particles were flake-like with an estimated size range of from about 200 nm to about 400 nm. The range of agglomerated nanoHA particles was from 98 nm to 845 nm in length, and from 43 nm to 300 nm in width. The crystal phase, as measured by XRD, showed that the major peaks were HA (see FIG. 2C). Hydroxyfluoroxyapatite [Ca₅(PO₄)₃(OH)] and calcium hydrogen phosphate hydroxide [Ca₉H(PO₄)₅(OH)] were also observed in the resulting nanopowder. Elemental analysis by EDS indicated that the elemental components of the nanoHA powder were P, O, and Ca (see FIG. 2D). In summary, the combination of XRD and EDS analysis indicates that the produced nanoHA powder was highly pure Hydroxyapatite.

6. Synthesis of Calcium Fluoride Phosphate Nanoparticles—Method A

In a sixth example, calcium fluoride phosphate nanoparticles were synthesized by precipitation under constant stirring conditions. The molar ratio of calcium to phosphorus was 1.67. Initially, 50 ml of 0.6 M H₃PO₄ was added in a dropwise manner to 50 ml of 1.0 M Ca(OH)₂ under stirring. 50 ml of a 1.0 M NaF solution was then added to the resulting solution, also under constant stirring, according to the reaction scheme below.

Ca(OH)₂+H₃PO₄+NaF=CaₓFₓ(PO₄)ₓ(OH)ₓ (3)

The reaction was performed for seven days with the pH maintained at 12.0. The resulting nanoparticles were purified by washing four times with deionized water. The purified nanoparticles were frozen at −80° C. and lyophilized.

7. Synthesis of Calcium Fluoride Phosphate Nanoparticles—Method B

In a seventh example, calcium fluoride phosphate nanoparticles were synthesized by precipitation under constant stirring conditions. The molar ratio of calcium to phosphorus was 1.67. Initially, 50 ml of 0.6 M H₃PO₄ was added in a dropwise manner to 50 ml of a 1.0 M CaCl₂ solution under constant stirring. 50 ml of a 2.0 M NH₄F solution was then added to the resulting solution, and the pH adjusted to 12.0 using 10 N NaOH solution, according to the reaction scheme below.

CaCl₂+H₃PO₄+NH₄F+NaOH=CaₓFₓ(PO₄)ₓ(OH)ₓ (4)

The reaction was performed for seven days with the pH maintained at 12.0. The resulting nanoparticles were purified by washing four times with deionized water. The purified nanoparticles were frozen at −80° C. and lyophilized.

8. Preparation of Nanocomposites

In another example, a nanoCaF₂/nanoDCPA nanocomposite was prepared by mixing a quantity of calcium fluoride nanoparticles (prepared in Examples 1 and 2) with a quantity of anhydrous dicalcium phosphate nanoparticles (prepared in Example 3).

A second nanocomposite (nanoCaF₂/nanoHA) was prepared by mixing a quantity of calcium fluoride nanoparticles (prepared in Examples 1 and 2) with a quantity of nano-Hydroxyapatite particles (prepared in Example 5).

9. Treatment Using Nanocomposite-Suspension Systems

In another example, the effectiveness of two nanocomposite-suspension systems was evaluated in a pilot study. Dentin disks were split into four quarters (Q1-Q4) after a pretreatment step. The pretreatment step comprised ultrasonic washing with 2% Micro-90 for about 3-5 minutes. The disks were then rinsed with deionized water for about 3 minutes, and then immersed in a 50% citric acid solution for about 2 minutes, before rinsing with deionized water and a final ultrasonic rinse for about 3 minutes.

A 4 wt. % suspension of nanoCaF₂/nanoHA (1:1, wt) was dropped onto the dentin surface of Q2-Q4 for a period of 2 minutes, after which the excess was removed. This procedure was repeated for a total of six times, after which the disks were rinsed in deionized water on a shaker (100 rpm) for one minute, followed by another rinse in deionized water. Sections Q2 and Q4 of each disk were treated a single time, whereas section Q3 was treated three times.

After treatment, section Q4 was coated with PERMASEAL, a commercially available methacrylate based resin.

Following treatment, sections Q2 and Q4 were immersed in human saliva (37° C.) for a period of 5 days. Section Q3 was immersed in human saliva for a period of 2 days, after the third treatment.

The occlusion of dentin tubules was evaluated by FESEM. Results indicate that approximately 95% of the dentin tubules were completely occluded after multiple treatments with the nanoCaF₂/nanoHA suspension (see FIG. 3C1). FIG. 3C2 illustrates partially occluded dentin tubules after multiple treatments.

For the sections exposed to a single treatment with the nanoCaF₂/nanoHA suspension, approximately 83% of the dentin tubules in the absence (FIG. 3B1) or presence (FIG. 3D1) or PERMASEAL, were completely occluded, and the remaining tubules (approximately 17%) were at least partially occluded (FIG. 3B2, 3D2).

FESEM analysis at high magnification (FIG. 3E, 3F) illustrates that nanoclusters of CaF₂ and HA infiltrated into the dentin tubules. EDS analysis confirmed that the deposited material comprised 0, F, P, and Ca.

10. Treatment Using Nanocomposite-Gel System

In another example, a bioadhesive aqueous carrier was prepared. Bioadhesive semisolid gels or toothpaste like paste were fabricated and compared according to the following formulations: Gel #1: 3% Gantrez AN119 (GAN119, MW 200,000, ISP gift), 0.5% hydroxyethylcellulose (HEC, 250 HHR, Ashland) and 0.5% polycarbophil (PC, Lubrizol); Gel #2: 0.5% HEC and 0.3% PC. Gel#3: 5-10% of GAN119 with 1% PC. Toothpaste like paste was fabricated based on the protocol of Colgate Total toothpaste. One buffered aqueous carrier (133 mM NaCl and 50 mM HEPES at pH 7.4) was also developed. 1.5 g of GAN119 was thoroughly dissolved in 50 mL of deionized H₂O under mechanical stirring before 250 mg of HEC was added in the preparation of Gel #1. Then the mixture was transferred onto an ointment slab and mixed with 250 mg of PC. For Gel#2, 250 mg of HEC was first thoroughly dissolved in 50 mL of 0.03 mol/L PBS (pH=6.8) under mechanical stirring and transferred on the ointment slab for mixing with 150 mg of PC. The Gel #3 was fabricated by stirring the 10% GAN119 in deionized water until it is completely dissolved. The pH of 10% GAN119 solution was adjusted to 7.4 before adding nanocomposite inside the solution. The nanocomposites were then added into the 10% GAN119 solution at percentage of 10%, 15% and 20% to form a GAN119-nanocomposite complex. Then 1% PC was then added into this complex. The bubbles in the gels were removed by vacuum desiccators under reduced pressure after they were transferred into an ointment amber jar. Then the gels were stored at 2-8° C. The gel were subsequently examined and determined by the mixing the nanoparticles and applying to portions (Q1-Q4) of the dentin disks. Surgical glue was also evaluated as a bioadhesive carrier. Section Q1 was exposed to a pretreatment, section Q2 was exposed to a single nanoCaF₂-gel treatment, section Q3 was exposed to a single nanoCaF2-gel treatment followed by immersion in human saliva (37° C.) for 24 hours, and section Q4 was exposed to a single nanoCaF2-gel treatment followed by immersion in Coca-Cola (room temperature) for 100 minutes.

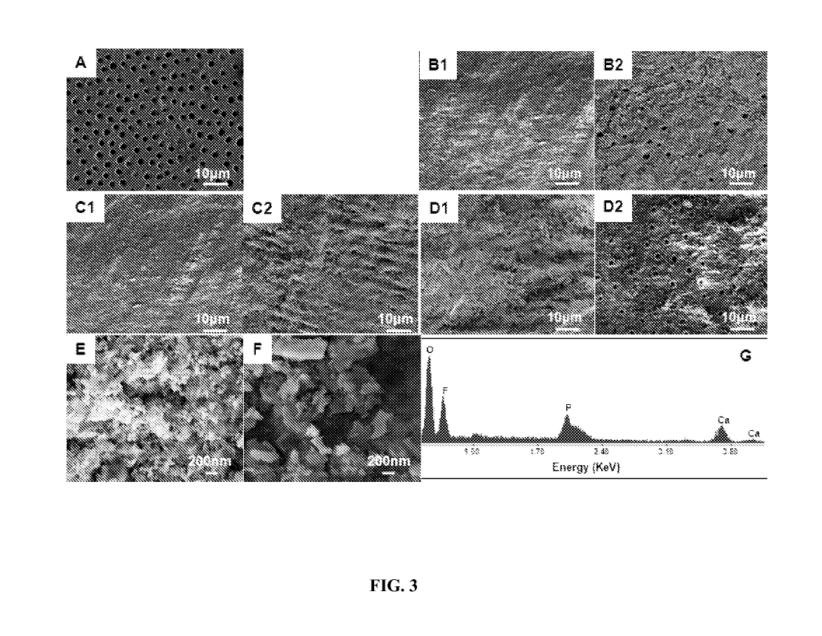

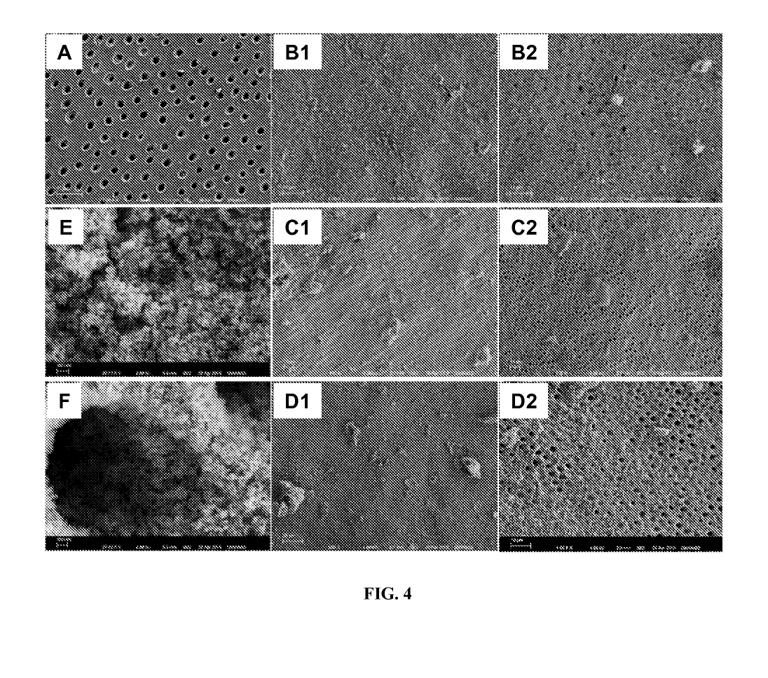

FESEM images of the resulting disk sections indicated that approximately 80-85% of the dentin tubules were completely or partially occluded after a single treatment (FIG. 4B1, 4B2). Similar results were observed for the single nanoCaF₂-gel treated disk followed by 24 hour immersion in human saliva (FIG. 4C1, 4C2). After treatment and immersion in Coca-Cola, approximately 50% of dentin tubules were partially or completely occluded (FIG. 4D1, 4D2). Electron microscopy indicated that the nanoclusters of CaF₂ particles had infiltrated the dentin tubules. FIGS. 4E and 4F showed the completely and partially occluded dentin tubules, respectively, at high magnification.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A composition comprising calcium fluoride nanoparticles, calcium fluoride phosphate nanoparticles, or a combination thereof.

2. The composition of claim 1, comprising hydroxyapatite nanoparticles.

3. The composition of claim 1, comprising dicalcium phosphate nanoparticles.

4. The composition of claim 1, comprising calcium fluoride nanoparticles having an average particle size of less than about 150 nm.

5. The composition of claim 1, comprising calcium fluoride nanoparticles having an average particle size of less than about 60 nm.

6. The composition of claim 1, wherein at least a portion of the particles are spherical or substantially spherical.

7. A nanocomposite comprising calcium fluoride nanoparticles and at least one of dicalcium phosphate nanoparticles, hydroxyapatite nanoparticles, or a combination thereof.

8. The nanocomposite of claim 7, wherein the amount of calcium fluoride nanoparticles is equal to or greater than the amount of dicalcium phosphate nanoparticles, hydroxyapatite nanoparticles, or the combination thereof.

9. (canceled)

10. A dentrifice comprising calcium, phosphate, and fluoride ions.

11. A dentrifice comprising the composition of claim 1.

12. A dentrifice comprising the nanocomposite of claim 7.

13. The dentifrice of claim 12, being capable of occluding at least a portion of exposed dentin tubules when topically applied to a tooth surface.

14. The dentrifice of claim 13, also capable of at least partially remineralizing tooth enamel.

15. A method for reducing dentin hypersensitivity, the method comprising contacting the composition of claim 1 with a tooth surface.

16. The method of claim 15, wherein the composition further comprises at least one of dicalcium phosphate nanoparticles, hydroxyapatite nanoparticles, or a combination thereof.
